(19) United States
(12) Patent Application Publication
(10) Pub. No.: US 2024/0197433 A1
(43) Pub. Date: Jun. 20, 2024

(54) REAL-TIME 3D ANATOMICAL MAPPING OF THE EYE

(71) Applicant: JOHNSON & JOHNSON SURGICAL VISION, INC., Irvine, CA (US)

(72) Inventor: Assaf Govari, Haifa (IL)

(21) Appl. No.: 18/387,494

(22) Filed: Nov. 7, 2023

Related U.S. Application Data
(60) Provisional application No. 63/433,799, filed on Dec. 20, 2022.

(51) Int. Cl. A61B 90/00; G06T 7/00; G06T 7/514; G06T 7/55; G06T 7/73; G06T 17/20; G06T 19/20

(52) U.S. Cl. CPC A61B 90/37; A61B 90/00; G06T 7/0012; G06T 7/514; G06T 7/55; G06T 7/73; G06T 17/20; G06T 19/20; A61B 2090/371; G06T 2207/10016; G06T 2207/10028; G06T 2207/10056; G06T 2207/30041; G06T 2210/41; G06T 2210/56; G06T 2219/2016; G06T 2219/2021

(57) ABSTRACT

A medical visualization apparatus includes two or more imaging devices and a processor. The two or more imaging devices are configured to acquire speckle images of an organ of a patient from multiple different directions. The processor is configured to (a) generate from the speckle images a three-dimensional (3D) point cloud of locations, (b) generate from the 3D point cloud of locations a 3D map of an anatomical surface of the organ, and (c) display the 3D map of the anatomical surface according to a requested gazing direction relative to the organ.

### CROSS-REFERENCE TO RELATED APPLICATIONS

The present application claims priority to and the benefit of U.S. Provisional Application No. 63/433,799, filed on Dec. 20, 2022, which is hereby incorporated by reference in its entirety.

### FIELD OF THE DISCLOSURE

The present disclosure relates generally to medical imaging, and particularly to real-time three-dimensional (3D) visual inspection and mapping of an eye.

### BACKGROUND OF THE DISCLOSURE

An eye is a complex optical system which collects light from the surrounding environment, regulates its intensity through a diaphragm, and focuses it through an adjustable assembly of lenses to form an image. To this end, the eye consists of a multi layered 3D eyeball made of various types of tissue, with each tissue having its unique material characteristics and features.

The present disclosure will be more fully understood from the following detailed description of the examples thereof, taken together with the drawings in which:

### BRIEF DESCRIPTION OF THE DRAWINGS

FIG. 1 is a schematic, pictorial view of an eye-inspection and surgery system comprising an apparatus for real-time generation and display of a 3D anatomical map of an eye, in accordance with an example of the present disclosure;

FIG. 2 is a schematic, pictorial view of a layout of the apparatus of FIG. 1 for real-time generation and display of a 3D anatomical map of an eye, in accordance with an example of the present disclosure;

FIG. 3 is a flow chart schematically illustrating a method for real-time generation and display of a 3D anatomical map of an eye using the apparatus of FIG. 1, in accordance with an example of the present disclosure; and

FIG. 4 is a flow chart schematically illustrating a method for real-time generation and overlaying a 3D video stream of the eye on the anatomical map of an eye, in accordance with an example of the present disclosure.

### DETAILED DESCRIPTION OF EXAMPLES

### Overview

A physician performing eye surgery usually looks at the surgical site within the eye through a microscope. While sometimes it would be more useful for the physician to be able to observe the surgery in real-time on a monitor in 3D, it is difficult to generate a quality video stream that can provide clinical-grade real-time 3D imaging.

Examples of the present disclosure that are described hereinafter generate a 3D anatomical map of a surgical site, such as a surgical site in an eye, where the 3D map can be video streamed (e.g., at 60 frames per second) and gazed at from any direction at will. The disclosed techniques are based on the assumption that simplified surfaces can represent anatomy in a meaningful way to the surgeon. For example, a technique is disclosed to generate a dome-shaped construction that shows the eye as it would be observed from the outside. The simplified 3D construction enables the display of a 3D anatomical map of the eye according to a requested gazing direction at the organ.

To generate and modify the 3D map in real time, the disclosed technique utilizes a dedicated algorithm optimized for use with a parallel processing capability. A master processor runs the algorithm using slave processors, so as to (i) generate a 3D point cloud of surface locations from the speckle images, and (ii) generate a 3D anatomical map from the 3D point cloud that can be viewed on a display from a requested gazing direction. Upon selecting a new gazing direction, the processor responds by displaying the requested real-time 3D map. The processor may comprise, for example, a Graphics Processing Unit (GPU) comprising multiple processing cores that operate in parallel.

In one example, to acquire image data for generating the 3D map, a speckle imaging set-up is provided, comprising a pair of largely diametrically opposing imaging devices, such as digital microscopes, each mounted on a mechanical stage to provide sub-pixel resolution of a 2D image. Typically, three digital microscopes provide 2D data for reconstructing the full 3D map of the eye, under the assumption that the eye is dome shaped. A 3D reconstruction of a dome can be also made with 2D data from two digital microscopes. In another example, the speckle imaging set-up comprises three digital microscopes, in a 120° angular separation layout.

One or more illumination sources (e.g., flashing LED lights) are used to generate speckled light. Each digital microscope acquires (e.g., captures) a speckle image projected from the surgical site by at least partially coherent illumination.

The position and the viewing direction of each of the optical speckle imaging devices are known. The master processor, running an algorithm suitable for parallel processing, analyzes the speckle images (i.e., starts the extraction of speckle coordinates) by distributing the captured rows of each 2D image data between different slave processors (e.g., in a GPU or TPU layout).

The slave processors generate a 3D point cloud of locations from the speckle images by (i) estimating lateral coordinates of each speckle (e.g., as projected on the pixel array of digital microscopes), and (ii) performing depth analysis of each speckle to estimate its depth coordinate. The slave processors perform depth analysis separately for each row in a parallel processing mode.

Having extracted the 3D coordinates, the master processor generates a 3D point cloud. In one example, the master processor aggregates the extracted speckle locations in space into a 3D point cloud data set with known coordinates of the data points of the 3D point cloud.

The master processor then reunites the rows and divides the 3D point cloud of locations into unit areas, such as into squares. The processor distributes the squares to other slave processors in the GPU, the latter computing the surface normal for each square, so as to geometrically characterize the 3D point cloud, which presumably lays on a variably curved surface.

Using the found orientation of unit areas, the master processor reconstructs a mesh approximation of a variably curved surface in 3D space from the oriented unit areas. For example, the master processor performs triangulation in each unit area (e.g., square), such as Delaunay triangulation, with the master processor connecting the corners of the squares with Delaunay triangulation to generate a triangular mesh approximation of the sought curved surface. The triangular mesh approximation is sufficiently accurate due to having the mesh edges coinciding in space with locations of a subset of the 3D point cloud.

The density of the 3D point cloud ensures spatial accuracy of the mesh representation of the actual anatomical surface at a level of hundred microns. Nevertheless, when smaller computing resources are required, the technique is still clinically useful in surgery even if mesh accuracy is lower, e.g., on the order of a 100-200 micron level of error in surface locations. Finally, the master processor displays the resulting surface (e.g., a dome-shaped reconstruction for the eye) on a display screen.

The physician can have means to select the gaze direction to view the curved surface, for instance by using an add-on for the finger to act as a virtual pointer, or using a touchscreen display. In response to selection, the processor moves (e.g., rotates) the displayed surface (i.e., 3D anatomical map), thereby allowing the physician to observe the surgical site in real time from the requested direction.

The master processor may graphically indicate different anatomical portions of the variably curved surface to enhance the clinical utility of the 3D anatomical map.

According to some example embodiments, the master processor overlays the eye model (e.g., 3D map) with a video stream of a 3D surface image of the eye. To this end, the processor computes from the video data acquired from digital microscopes a video projection onto the entire eye surface. Superimposing video stream onto a curved surface can involve combing video streams acquired from different point of views while applying necessary mathematical transformations.

The disclosed 3D anatomical mapping technique generates and displays an approximation of surface anatomy with spatial precision of tens of microns when viewed from an arbitrary gazing direction. This may allow the performance of surgeries, such as eye surgeries, more easily, more effectively, and with fewer hazards.

### System Description

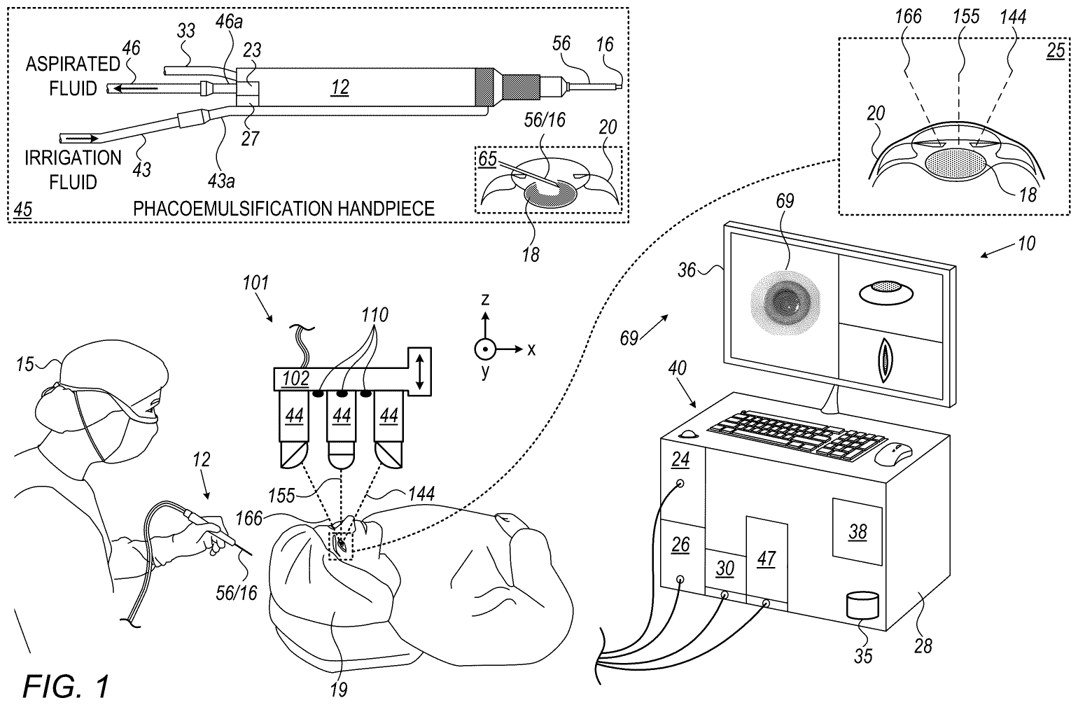

FIG. 1 is a schematic, pictorial view of an eye-inspection and surgery system 10 comprising an apparatus 101 for real-time generation and display of a 3D anatomical map of an eye, in accordance with an example of the present disclosure.

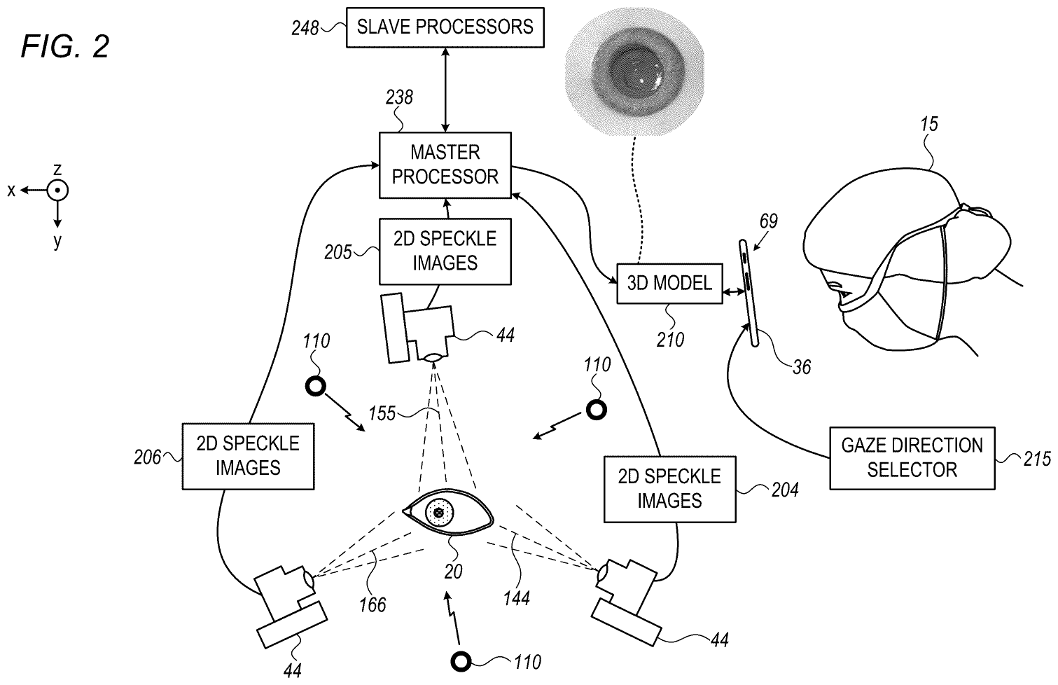

System 10 has three digital microscopes 44. The trio is equidistantly arranged over a circular parameter in a 120° point-of-view layout directed at an eye 20 of a patient 19. A top view of the circular arrangement is provided in FIG. 2.

The digital microscope trio is rigidly mounted on a stage 102 capable of accurate Z-axis motion, and optionally, lateral XY motion (e.g., on a stage suspended from a ceiling). The resolution of stage 102 motion is in the order of a pixel size of the digital microscopes.

Digital microscopes 44 are mounted to have optical imaging axes 144, 155 and 166. As inset 25 shows, directions 144/155/166 are aligned so as to provide an unobstructed optical path for each of the microscopes 44 to view a lens 18 of eye 20. Three digital microscopes are used so as to always have at least two digital microscopes that are not obstructed by the handpiece 12 in the surgical site.

Stage 102 and digital microscopes 44 are controlled by a processor 38 that may vary a lateral location in space at which directions 144/155/166 cross each other and/or varying depth of imaging along directions 144/155/166. Stage 102 enables fine tuning the alignment of the microscope-trio with respect to eye lens 18.

Each microscope may have a variable focus length along its respective optical axis. Digital microscopes 44 are mounted on a Z-Stage that provides adjustment of the vertical distance of the digital microscopes from the eye. Optionally, stage 102 also provides moving the digital microscopes in the X-Y direction. In some examples, stage 102 is optional and there is no need to move the digital microscopes. Instead, the patient's head is aligned to the field of view of the digital microscopes. If a stage 102 is used a cable is used to convey control signals to move the stage. A multiwire cable is used to convey signals from digital microscopes 44 to processor 38 and to provide power to flash LED illumination sources 110.

System 10 generates a 3D anatomical map 69 of eye 20 (e.g., of a surgical site in eye 20) from 2D image data captured by the digital microscopes. The processor video streams the captured video in 3D by overlaying the video on the 3D map 69 of the eye on a display 36 in console 28. The 3D map is video streamed to the displays. Physician 15 can therefore view the surgical site as a video of the surgical site overlayed on a rendered 3D map. The 3D map and video are updated in real time and the physician can choose to observed the rendered 3D construction from any arbitrary direction by selecting a gaze direction, for instance by using a mouse or a touchscreen type of display to rotate the displayed 3D map 69 to a new orientation.

System 10 may present other results of a diagnostic and/or therapeutic procedure on display 36. System 10 may receive user-based commands via a user interface 40. User interface 40 may be combined with a touchscreen graphical user interface of display 36.

Further seen in FIG. 1 is a phacoemulsification probe 12 of system 10. In the pictorial view and in the orthographic side view inset 45, a phacoemulsification probe 12 (e.g., a handpiece) comprises a distal end 112 comprising a needle 16 and a coaxial irrigation sleeve 56 that at least partially surrounds needle 16 and creates a fluid pathway between the external wall of the needle and the internal wall of the irrigation sleeve, where needle 16 is hollow to provide an aspiration channel.

As sub-inset 65 shows, needle 16 is configured for insertion into lens capsule 18 of eye 20 of a patient 19 by physician 15 to remove a cataract.

During the phacoemulsification procedure a processor-controlled irrigation pump 24, comprised in a console 28, pumps irrigation fluid from an irrigation reservoir (not shown) to the irrigation sleeve 56 to irrigate the eye. The fluid is pumped via an irrigation tubing line 43 running from console 28 to an irrigation channel 43a of probe 12.

Eye fluid and waste matter (e.g., emulsified parts of the cataract) are aspirated via hollow needle 16 to a collection receptacle (not shown) by a processor-controlled aspiration pump 26, also comprised in console 28, using aspiration tubing line 46 running from aspiration channel 46a of probe 12 to console 28.

Channels 43a and 46a are coupled respectively with irrigation line 43 and aspiration line 46. Pumps 24 and 26 may be any pump known in the art (e.g., a peristaltic pump). Using sensors 27 and/or 23 coupled respectively to irrigation and aspiration channels 43a and 46a, processor 38 controls a pump rate of irrigation pump 24 and aspiration pump 26 to maintain intraocular pressure (IOP) within prespecified limits.

Console 28, which is part of system 10, comprises a piezoelectric drive module 30 that drives needle 16 to vibrate in a resonant vibration mode that is used to break a cataract into small pieces during a phacoemulsification procedure. To this end, drive module 30 is coupled with the piezoelectric crystal inside handpiece 12 (not shown) using electrical wiring running in a cable.

Some or all of the functions of processor 38 may be combined in a single physical component or, alternatively, implemented using multiple physical components. In some examples, at least some of the functions of processor 38 may be carried out by suitable software stored in a memory 35 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

The apparatus shown in FIG. 1 may include further elements, which are omitted for clarity of presentation. For example, physician 15 may hold a control handle which can command processor 38. Physician 15 may use surgical tools and/or apply medications, which are also not shown in order to maintain clarity and simplicity of presentation. Processor 38 may receive user-based commands via a user interface 40, which may include setting a vibration mode of needle 16, and setting or adjusting an irrigation and/or aspiration. Typically, the physician uses a foot pedal (not shown) as a means of control. For example, foot pedal position one activates only irrigation, pedal position two activates both irrigation and aspiration, and pedal position three adds needle 16 vibration. Additionally, or alternatively, processor 38 may receive user-based commands from controls located in a handpiece 12.

### Real-Time 3D Anatomical Mapping of the Eye

FIG. 2 is a schematic, pictorial view of a layout of apparatus 101 of FIG. 1 for real-time generation and display of a 3D anatomical map of an eye, in accordance with an example of the present disclosure.

As seen, digital microscopes 44 are arranged in space to have at least two unobstructed views of views 144/155/166 of eye 20, while capturing respective 2D speckle images 204/205/206 of eye 20 that are flash illuminated by LEDs 110. An obstruction to one of the digital microscopes 44 can happen due to, for example, handpiece 12 being the way. To best avoid obstruction, digital Microscopes 44 are angularly separated by 120° to ensure complete and uniform coverage of eye 20 dome-like surface. The result is a sufficiently spatially uniform and dense point cloud speckle data. In principle, two microscopes (e.g., arranged diametrically opposed when projected to the XY plan) can also be sufficient in that regard, albeit with some limitations on coverage at the edges of fields of views.

The 2D speckle images are received in a master processor, such as processor 38. The processor divides each speckle image into unit areas and performs depth analysis of each unit area. The processor estimates orientation of each unit area. Then the processor generates a 3D point cloud from the speckle images of locations representing surfaces of the eye that reflect the flash illumination.

From the 3D point cloud data, using the depth and orientation of unit areas, the processor reconstructs a mesh approximation of a surface in 3D space, the mesh edges corresponding to a subset of the 3D point cloud. Using the mesh, the processor generates a 3D map (e.g., 3D model) 210 of an anatomical surface of eye 20. Finally, the processor displays the 3D map in a form of an eye map 69 of the anatomical surface of eye 20.

As seen, physician 15 can change a gaze direction to view eye map 69 using a gaze direction selector 215. Selector 215 can be a mouse or a touch screen connected to the display 36.

The example unit shown in FIG. 2 is chosen purely for the sake of conceptual clarity and simplicity. For example, using additional controls, physician 15 may choose to switch between eye map 69 to information from any of one or more modalities available in the surgical theater.

### Method for Real Time 3D Anatomical Mapping of the Eye

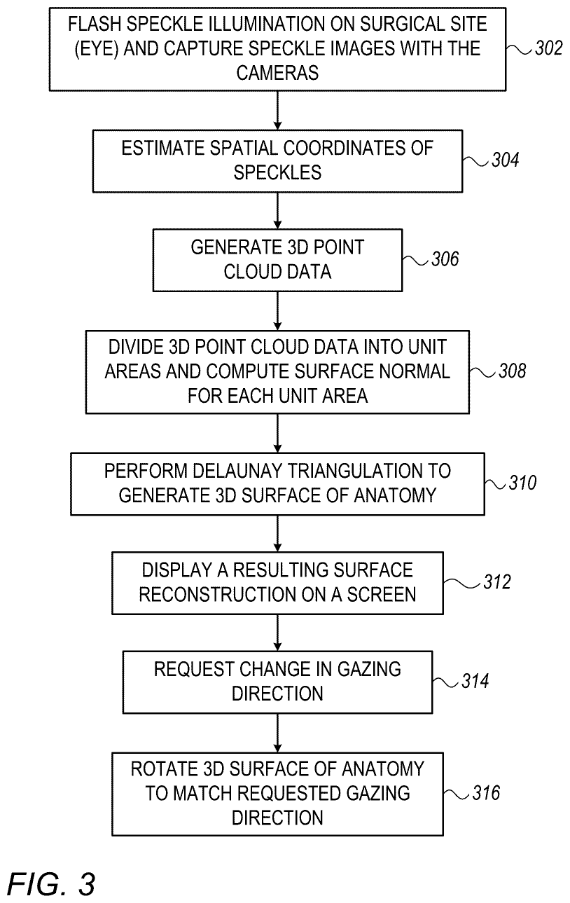

FIG. 3 is a flow chart schematically illustrating a method for real-time generation and display of a 3D anatomical map of an eye using apparatus 101 of FIG. 1, in accordance with an example of the present disclosure. The algorithm, according to the presented example, carries out a process that begins with physician 15 operating system 10 (e.g., processor 38) to perform an eye surgery by initiating the activation of apparatus 101. In response, at speckle imaging step 302, apparatus 101 illuminates flash coherent light on the surgical site (e.g., the eye), while the imaging devices (e.g., the trio of digital microscopes mounted on respective microscopes) capture 2D speckle images.

The speckle image data is received at a master processor 238, which utilizes slave processors 248 to estimate the coordinates in space (i.e., spatial coordinates) of the speckles, at speckle location extraction step 304. In one example, a GPU is used to (i) capture the images with the digital microscopes, (ii) Distribute the captured rows of image data between different processors in the GPU to perform depth analysis separately for each row with parallel processing.

Next, at 3D cloud data generation step 306, master processor 238 generates a 3D point cloud with known coordinates of the data points of the 3D point cloud. In one example, this is accomplished when e master processor aggregates the estimated speckle locations in space, which are received from slave processors 248.

Next, master processor 238 divides the 3D point data into unit areas and then distributes the unit areas among slave processors 248 that calculate surface normal for each unit area, at a local surface orientation computation step 308.

After receiving local surface orientations, master processor 238 generates a triangular mesh approximation from the unit areas of the variably curved surface, in a 3D surface anatomy map generation step 310.

At a 3D anatomical map displaying step 312, processor 238 displays the surface reconstruction on display 36.

At request change of gazing direction step 314, physician 15 may see the 3D anatomical map from another direction with commands (215) to processor 238 to, for example, attempt to rotate the 3D surface on a touchscreen type of display 36. Another option to change a gaze direction is to select a requested direction from a list, such as one comprising anterior/posterior, inferior/superior, lateral left/right directions, or combinations thereof.

In response to a request of step 314, processor 238 reconstructs and displays the 3D anatomical map as seen from the requested gazing direction, which the processor does at a video rate, at a 3D gazing direction changing step 316.

The example flow chart shown in FIG. 3 is chosen purely for the sake of conceptual clarity. For example, additional inspection steps, such as noninvasive measurements (intra-ocular pressure, eye temperature), may also be performed while examining the 3D anatomical map.

### Overlaying Video Image on Anatomical Surface Reconstruction

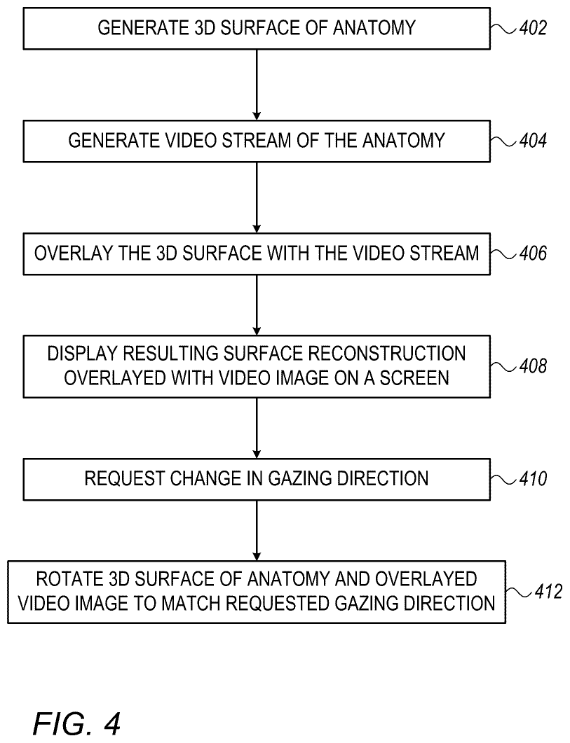

FIG. 4 is a flow chart schematically illustrating a method for real-time generation and overlaying a 3D video stream of the eye on the anatomical map of an eye, in accordance with an example of the present disclosure. In one example, the process is performed by the apparatus of FIG. 1. The algorithm, according to the presented example, carries out a process that begins with physician 15 operating system 10 (e.g., processor 38) to generate a 3D surface anatomy map, in 3D map generation step 402 (this step is also described in step 310 of FIG. 3).

At video stream generation step 404, the processor generates a video stream of the anatomy. In one example, the video stream is a 3D surface image of an eye undergoing surgery.

Next, the processor overlays the video stream onto the 3D surface, at an overlaying step 406. The video images are computed so that they can be overlayed on a curved surface.

At a displaying step 408, the processor displays the 3D anatomical map overlayed with the video stream. The physician can select which layer to see, or apply a semitransparent mode, where both layers (the anatomical surface and the video) are visible.

At request change of gazing direction step 410, physician 15 may see the 3D anatomical map and/or the overlayed video stream from another direction.

In response to a request of step 410, processor 238 performs 3D rendering to show on display 36 based on the gazing direction selected, at a 3D gazing direction changing step 412.

### EXAMPLES

### Example 1

A medical visualization apparatus (101) includes two or more imaging devices (44) and a processor (38). The two or more imaging devices are configured to acquire speckle images of an organ of a patient from multiple different directions (144, 155, 166). The processor is configured to (a) generate from the speckle images a three-dimensional (3D) point cloud of locations, (b) generate from the 3D point cloud of locations a 3D map (69) of an anatomical surface of the organ, and (c) display the 3D map (69) of the anatomical surface according to a requested gazing direction relative to the organ.

### Example 2

The apparatus (101) according to example 1, wherein the processor (38) is configured to generate a video stream of the organ and overlay the video stream on the 3D map (69).

### Example 3

The apparatus (101) according to any of examples 1 and 2, wherein the processor (38) is configured to generate the 3D map (69) of the anatomical surface by performing at least the steps of (i) estimating lateral coordinates of speckles in the speckle images, (ii) performing depth analysis of the speckles to estimate depth coordinates of the speckles, (iii) aggregating estimated spatial locations of the speckles into a 3D point cloud data set, (iv) dividing the 3D point cloud data set into unit areas, and estimate respective orientations of the unit areas, and (v) using the orientations of the unit areas, reconstructing from the 3D point cloud a mesh approximation of a variably curved surface in 3D space.

### Example 4

The apparatus (101) according to any of examples 1 through 3, wherein the processor (38) is further configured to graphically indicate different anatomical portions of the variably curved surface.

### Example 5

The apparatus (101) according to any of examples 1 through 4, wherein the mesh approximation is a triangular mesh.

### Example 6

The apparatus (101) according to any of examples 1 through 5, wherein edges of the mesh approximation are locations of a subset of the data points of the 3D point cloud.

### Example 7

The apparatus (101) according to any of examples 1 through 6, wherein the imaging devices (44) are configured to acquire a stream of the speckle images at a video rate, and wherein the processor (38) is configured to generate and display the 3D map (69) at the video rate.

### Example 8

The apparatus (101) according to any of examples 1 through 7, wherein the processor (38) is further configured to, in response to a request to change the gazing direction, display the 3D map (69) of the anatomical surface as viewed from a new direction.

### Example 9

The apparatus (101) according to any of examples 1 through 8, wherein the processor (38) is configured to respond to the request at the video rate.

### Example 10

The apparatus (101) according to any of examples 1 through 9, wherein the imaging devices (44) comprise digital microscopes.

### Example 11

The apparatus (101) according to any of examples 1 through 10, wherein the organ is an eye (20).

### Example 12

A medical visualization method includes acquiring speckle images of an organ of a patient from multiple different directions (144, 155, 166) using two or more imaging devices (44). A three-dimensional (3D) point cloud of locations is generated from the speckle images. A 3D map (69) of an anatomical surface of the organ is generated from the 3D point cloud of locations. The 3D map (69) of the anatomical surface is displayed according to a requested gazing direction relative to the organ.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A medical visualization apparatus, comprising:
two or more imaging devices configured to acquire speckle images of an organ of a patient from multiple different directions; and
a processor, which is configured to:
generate from the speckle images a three-dimensional (3D) point cloud of locations;
generate from the 3D point cloud of locations a 3D map of an anatomical surface of the organ; and
display the 3D map of the anatomical surface according to a requested gazing direction relative to the organ.

2. The apparatus according to claim 1, wherein the processor is configured to generate a video stream of the organ and overlay the video stream on the 3D map.

3. The apparatus according to claim 1, wherein the processor is configured to generate the 3D map of the anatomical surface by performing at least the steps of:
estimating lateral coordinates of speckles in the speckle images;
performing depth analysis of the speckles to estimate depth coordinates of the speckles;
aggregating estimated spatial locations of the speckles into a 3D point cloud data set;
dividing the 3D point cloud data set into unit areas, and estimate respective orientations of the unit areas; and
using the orientations of the unit areas, reconstructing from the 3D point cloud a mesh approximation of a variably curved surface in 3D space.

4. The apparatus according to claim 3, wherein the processor is further configured to graphically indicate different anatomical portions of the variably curved surface.

5. The apparatus according to claim 3, wherein the mesh approximation is a triangular mesh.

6. The apparatus according to claim 3, wherein edges of the mesh approximation are locations of a subset of the data points of the 3D point cloud.

7. The apparatus according to claim 1, wherein the imaging devices are configured to acquire a stream of the speckle images at a video rate, and wherein the processor is configured to generate and display the 3D map at the video rate.

8. The apparatus according to claim 1, wherein the processor is further configured to, in response to a request to change the gazing direction, display the 3D map of the anatomical surface as viewed from a new direction.

9. The apparatus according to claim 8, wherein the processor is configured to respond to the request at the video rate.

10. The apparatus according to claim 1, wherein the imaging devices comprise digital microscopes.

11. The apparatus according to claim 1, wherein the organ is an eye.

12. A medical visualization method, comprising:
acquiring speckle images of an organ of a patient from multiple different directions using two or more imaging devices;
generating from the speckle images a three-dimensional (3D) point cloud of locations;
generating from the 3D point cloud of locations a 3D map of an anatomical surface of the organ; and
displaying the 3D map of the anatomical surface according to a requested gazing direction relative to the organ.

13. The method according to claim 12, and comprising generating a video stream of the organ and overlaying the video stream on the 3D map.

14. The method according to claim 12, wherein generating the 3D map of the anatomical surface comprises performing at least the steps of:
estimating lateral coordinates of speckles in the speckle images;
performing depth analysis of the speckles to estimate depth coordinates of the speckles;
aggregating estimated spatial locations of the speckles into a 3D point cloud data set;
dividing the 3D point cloud data set into unit areas, and estimate respective orientations of the unit areas; and
using the orientations of the unit areas, reconstructing from the 3D point cloud a mesh approximation of a variably curved surface in 3D space.

15. The method according to claim 14, and comprising graphically indicating different anatomical portions of the variably curved surface.

16. The method according to claim 14, wherein the mesh approximation is a triangular mesh.

17. The method according to claim 14, wherein edges of the mesh approximation are locations of a subset of the data points of the 3D point cloud.

18. The method according to claim 12, wherein the imaging devices are configured to acquire a stream of the speckle images at a video rate, and wherein the processor is configured to generate and display the 3D map at the video rate.

19. The method according to claim 12, and comprising, in response to a request to change the gazing direction, displaying the 3D map of the anatomical surface as viewed from a new direction.

20. The method according to claim 19, and comprising responding to the request at the video rate.

21. The method according to claim 12, wherein the imaging devices comprise digital microscopes.

22. The method according to claim 12, wherein the organ is an eye.
